(19) United States
(12) Patent Application Publication
(10) Pub. No.: US 2025/0295571 A1
(43) Pub. Date: Sep. 25, 2025

(54) COMPOSITIONS AND METHODS FOR STIMULATION OF HAIR GROWTH

(71) Applicant: THE REGENTS OF THE UNIVERSITY OF CALIFORNIA, OAKLAND, CA (US)

(72) Inventors: Maksim PLIKUS, IRVINE, CA (US); Yingzi LIU, IRVINE, CA (US); Christian Fernando GUERRERO-JUAREZ, IRVINE, CA (US)

(73) Assignee: THE REGENTS OF THE UNIVERSITY OF CALIFORNIA, OAKLAND, CA (US)

(21) Appl. No.: 18/864,412

(22) PCT Filed: May 9, 2023
(86) PCT No.: PCT/US23/66770

§ 371 Date: Nov. 8, 2024

Related U.S. Application Data
(60) Provisional application No. 63/364,389, filed on May 9, 2022.

(51) Int. Cl. A61K 8/64; A61Q 7/00

(52) U.S. Cl. CPC A61K 8/64; A61Q 7/00; A61K 2800/594; A61K 2800/805; A61K 2800/91

(57) ABSTRACT

Proteins, peptides, nucleic acid molecules, compositions, and methods are provided for stimulating hair growth. Described herein is a composition comprising a Signal Peptide, CUB Domain and EGF Like Domain Containing (SCUBE) protein; a C-terminus peptide thereof (SCUBE-C); a synthetic SCUBE peptide (SCUBE-PP); or a nucleic acid sequence encoding SCUBE, SCUBE-C, or SCUBE-PP; and, optionally, a pharmaceutically acceptable excipient. In some embodiments, the SCUBE protein is a SCUBE3 protein; a C-terminus peptide thereof (SCUBE3-C); a synthetic SCUBE3 peptide (SCUBE3-PP). These materials and methods can be used to treat alopecia, to stimulate and accelerate hair regrowth after transplant, and to induce human hair growth.

This application claims benefit of U.S. provisional patent application No. 63/364,389, filed May 9, 2022, the entire contents of which are incorporated by reference into this application.

### REFERENCE TO A SEQUENCE LISTING

The content of the XML file of the sequence listing named “UCI012_seq”, which is 11 kb in size, created on Apr. 30, 2023, and electronically submitted herewith the application, is incorporated herein by reference in its entirety.

### BACKGROUND

Hair loss, or alopecia, affects a large fraction of the general population. For example, androgenic alopecia (hormonally induced common baldness) affects up to 50% of women and 80% of men, and alopecia areata (autoimmune form of hair loss) affects 2% of the general population (Alessandrini et al., 2021). Alopecia poses significant psychosocial distress to affected individuals. Although the pharmacological treatment for alopecia is highly desired and generates billions of dollars annually worldwide, the therapeutic options approved by the United States Food and Drug Administration (FDA) are limited to oral finasteride, topical minoxidil and topical corticosteroids (Cardoso et al., 2021). But all these treatments are either of limited efficacy or with high risk of adverse side effects. For example, topical application of minoxidil lotion has the disadvantage of limited efficiency, and many patients are poorly compliant because of the necessity to apply the medication twice a day and to remain taking medication constantly for its effect to remain in place. Adverse effects are the major concerns of oral finasteride and corticosteroids, including post-finasteride syndrome and immunosuppression respectively. Hence, developing novel treatment of hair loss is highly sought after and of great commercial benefit.

Knowledge of hair growth cycle has shed light on developing new treatment strategies for hair loss. The hair follicle (HF) is a complex mini-organ in the skin that sustains cyclic hair regrowth over repeated hair cycles, consisting of the consecutive phases of active growth (anagen), regression (catagen) and rest (telogen). Hair growth phase is fueled by hair follicle stem cell (HFSC), which are sustained and regulated by interaction with its local environment, or ‘niche’. Dermal papilla cells (DPs), the specialized fibroblasts embedded in the hair bulb at the base of the HF, constitute the critical HFSC niche. Signaling from DPs is required for hair development during embryogenesis and for cyclic hair growth in adults. DP dysfunction can disrupt human hair growth, resulting in hair loss conditions such as androgenetic alopecia (Chen et al., 2020). Given the critical necessity of DPs in hair growth and its great transitional potential for treating hair conditions, better understanding of the molecular biology of DPs can lead to the discovery of novel molecular targets for hair growth stimulation.

There remains a need for novel materials and methods for stimulation of hair growth.

### SUMMARY

The methods described herein provide proteins and peptides, as well as compositions, nucleic acid molecules, and methods for delivering same, to stimulate hair growth. These materials and methods can be used to treat alopecia, to stimulate and accelerate hair regrowth after transplant, and to induce human hair growth. Data presented in Example 1 below show that SCUBE3 can prominently induce new hair growth even in aged (20-month-old) mice. Moreover, human data show that SCUBE3 in human scalp hair follicle is expressed in the same cell type as in mouse-dermal papilla fibroblasts, thus in a conserved expression location. In addition, in a human-on-mouse xenograft model, SCUBE3 induces human hair growth.

Described herein is a composition comprising a Signal Peptide, CUB Domain and EGF Like Domain Containing (SCUBE) protein; a C-terminus peptide thereof (SCUBE-C); a synthetic SCUBE peptide (SCUBE-PP); or a nucleic acid sequence encoding SCUBE, SCUBE-C, or SCUBE-PP; and, optionally, a pharmaceutically acceptable excipient. In some embodiments, the SCUBE protein is a SCUBE3 protein (SEQ ID NO: 1); a C-terminus peptide thereof (SCUBE3-C; SEQ ID NO: 2); a synthetic SCUBE3 peptide (SCUBE3-PP). In some embodiments, the composition comprises a nucleic acid sequence encoding SCUBE3,SCUBE3-C, or SCUBE3-PP. In some embodiments, the nucleic acid sequence further comprises a promoter and/or other regulatory elements in operable linkage with the SCUBE protein or peptide encoding sequence. Other examples of a SCUBE protein include

SCUBE1 (Protein Accession: Q8IWY4; SEQ ID NO: 3) and SCUBE2 (Protein Accession: Q9NQ36; SEQ ID NO: 5). Use of SCUBE1, SCUBE2, and/or SCUBE3 proteins or peptides (e.g., SCUBE1-C (SEQ ID NO: 4), SCUBE1-PP. SCUBE2-C (SEQ ID NO: 6), SCUBE2-PP) is contemplated, optionally in combination.

In some embodiments, the SCUBE3-C comprises 3 cysteine-rich domains and one CUB domain (Complement proteins C1r/C1s, Uegf and Bmp1). In some embodiments, the SCUBE3-C peptide is produced by proteolytic cleavage of SCUBE3 with a protease. In some embodiments, the protease is one or more matrix metalloprotease (MMP) enzymes. In some embodiments, the MMP enzyme is synthetic. In some embodiments, the composition further comprises an MMP enzyme. In some embodiments, the MMP enzyme is selected from MMP2 and MMP9. In some embodiments, the SCUBE3-PP comprises 3 cysteine-rich domains and a CUB domain, a CUB domain and one cysteine-rich domain; or a CUB domain and two cysteine-rich domains.

In some embodiments, the 3 cysteine-rich domains have the amino acid sequence: GTKCVSCP QGTYYHGQTE QCVPCPAGTF QEREGQLSCD LCPGSDAHGP LGATNVTTCA GQCPPGQHSV DGFKPCQPCP RGTYQPEAGR TLCFPCGGGL TTKHEGAISF QDCDTKVQCS PGHYYNTSIH RCIRCAMGSY QPDFRQNFCS RCPGNTSTDF DGSTSVAQCK NRQ (SEQ ID NO: 7). In some embodiments, the CUB domain has the amino acid sequence: CGGELGE FTGYIESPNY PGNYPAGVEC IWNINPPPKR KILIVVPEIF LPSEDECGDV LVMRKNSSPS SITTYETCQT YERPIAFTAR SRKLWINFKT SEANSARGFQ IPYVTYDEDY EQLVEDIVRD GRLYASENHQ EILKDKKLIK AFFEVLAHPQ NYFKYTEKHK EMLPKSFIKL LRSKVSSFLR PYK (SEQ ID NO: 2).

Also described is a method of promoting hair growth in a subject. Additionally provided is a method of treating alopecia in a subject. In some embodiments, these methods comprise intradermal microinjection of a SCUBE composition as described herein. In some embodiments, these methods comprise topical skin administration of a SCUBE composition as described herein. In some embodiments, the topical skin administration is provided following an increase in skin permeability. In some embodiments, the increase in skin permeability occurs by micro-needling.

In some embodiments, the subject is a mammalian subject. In some embodiments, the subject is human. In some embodiments, the subject suffers from alopecia. In some embodiments, the alopecia is androgenic alopecia, female pattern hair loss, alopecia areata, traction alopecia, alopecia totalis, cicatricial alopecia, and/or alopecia universalis. In some embodiments, the method generates new hair follicles, accelerates hair regrowth after a hair transplantation, chemotherapy, and/or increases the size of existing hair follicles.

### BRIEF DESCRIPTION OF THE DRAWINGS

FIGS. 1A-1I. Hyper-activated hair regeneration in mice with dermal Hedgehog activation in LeprB-targeted dermal papillae. (1A) Co-expression patterns of pan-isoform Lepr mRNA and GFP reporter in dorsal LeprB-Cre;mTmG mouse skin at P50. Co-localization is seen in DPs. (1B) Left: Schematic diagram of LeprB-CreER transgenic mouse generation. Right: wholemount showing tdTomato expression in LeprB-Cre;tdTomato dorsal skin at P55. tdTomato fluorescence was observed in almost all DPs at all timepoints and in scattered vessel-like structures. (1C) Time course analysis of dorsal hair growth patterns in representative LeprBCre;SmoM2^{+/-}-mutant (bottom) and LeprB-Cre control mice (top) between days P46-84. Mice were shaved after each round of photography. Hair growth (dark regions, marked) is seen only in the mutant mouse. (1D) Unsupervised hierarchical clustering of all cells from control (k=18.496) and mutant (k=16,792) samples reveals 17 main cell types, which are color-coded and annotated at the bottom. (Color-coding shown in Liu Y, et al. Dev Cell. 2022 Jul. 25,57 (14): 1758-1775.) Cycling EPI—cycling interfollicular keratinocytes, Krt14⁺EPI—Krt14⁺interfollicular keratinocytes, Krt 10⁺EPI—Krt10⁺ interfollicular keratinocytes, lvl⁺ EPI—lvl⁺ interfollicular keratinocytes, Upper HF—upper hair follicle keratinocytes, Lower HF—lower hair follicle keratinocytes, SG—sebaceous gland cells/sebocytes, FIB—dermal fibroblasts, DP/DP-like—dermal papilla/dermal papilla-like fibroblasts, FA-FIB—fibro-adipogenic fibroblasts, MUSC—muscle cells, ENDO—endothelial cells, LYM—lymphatic endothelial cells, NAEC—neuron-associated endothelial cells, IMM—immune cells, SCH—Schwann cells, and MEL—melanocytes. (1E) Feature plots of Scube3 expression in control vs. mutant DP/DPL fibroblasts at P46. Mutant specific DPL cells were lined out. (1F) Activity heatmap of selected regulons across all DP subclusters, with control and mutant cells identified separately. (1G) Trackplot showing expression patterns of selected target genes of Gli1^{Reg(+)} regulon across all DP subclusters. Genes are annotated on the right. Gene expression levels are represented on a per-cell basis by track height and are accompanied by their specific motifs on the right. AUC—area under the curve; GEX—gene expression. (1H) Expression of selected Gli1^{Reg(+)} target genes—Scube3 in control (top) and mutant skin (bottom) at P46. DPs are magnified on the right. (1I) Scube3 mRNA in control (left) and mutant HFs (right) at P50. Tissues were counterstained with DAPI in 1A, 1H, 1I. Scale bars: 1A, 1H—100 μm, 1I—50 μm.

FIGS. 2A-2I. SCUBE3 induces hair growth. (2A) Expression of pan-isoform Scube3 mRNA in dorsal mouse skin at indicated postnatal time points. (2B) Time course analysis of dorsal hair growth patterns in representative control (top) and Scube3^{-/-} mice (bottom) between days P24-30. Mice were shaved before each round of photography. (2C) Representative images of microinjection sites for BSA (top) and rhSCUBE3 (bottom). Affi-gel blue agarose beads are visible. Newly growing anagen HFs are pigmented. (2D) Quantification of induced anagen HFs in response to rhSCUBE3 on day 14. n=7; *** P<0.0002. (2E) Representative images of microinjection sites for BSA (left) and rhSCUBE3 (right) on 20-month-old mice. Affi-gel blue agarose beads are visible. Newly growing anagen HFs are pigmented. (2F) Quantification of induced anagen HFs in aged mice on day 14 after protein injection. n=9; * P<0.0001. (2G) Expression of K14, KI67 and SCUBE3 mRNA in human anagen occipital scalp HF. SCUBE3 mRNA is specifically and highly expressed in DP and adjacent dermal sheath cup. (2H) Representative images of microinjection sites for BSA and rhSCUBE3 in human occipital scalp HF-on-SCID mouse xenograft model. (2I) Scattered plot of human and mouse anagen HF quantification. Striped dots represent rhSCUBE3-injected samples and solid dots represent BSA-injected samples. Tissue sections were counterstained with DAPI in 2A, 2G. Scale bars: 2A, 2G—50 μm.

FIG. 3. Schematic illustration of Scube3 flox/flox mouse design.

FIG. 4. Schematic illustration of TetO-Scube3 mouse design.

FIGS. 5A-5B. SCUBE1, SCUBE2, SCUBE3 CUB domain analysis and expression. (5A) Protein BLAST analysis of CUB domain of SCUBE1 (SEQ ID NO: 4), SCUBE2 (SEQ ID NO: 6) and SCUBE3 (SEQ ID NO: 2) protein sequences. SCUBE1 and SCUBE2 exhibit 83.25% and 82.20% homology with SCUBE3 in the CUB domain respectively. (5B) Feature plots of Scube1, Scube2 and Scube3 expression in control (LeprB-Cre;mTmG) and mutant (LeprB-Cre;SmoM2^{+/-};mTmG) fibroblasts at P46. DP/DPL fibroblasts were lined out. Scube1, Scube2 and Scube3 showed a similar expression pattern among fibroblasts.

### DETAILED DESCRIPTION

Described herein is a new molecular strategy of treating hair loss conditions, via activating SCUBE3 signaling in the hair-bearing skin of affected subjects. Target conditions include various forms of hair loss, including but not limited to androgenetic alopecia and alopecia areata. In one aspect, the activator of hair growth is naturally occurring human SCUBE3 (Signal Peptide, CUB Domain And EGF Like Domain Containing 3) protein, produced as pure recombinant protein. In another aspect, the activator of hair growth is purified C-terminus SCUBE3 peptide (SCUBE3-C), produced synthetically, recombinantly, or by proteolytic cleavage of natural SCUBE3 with one or several matrix metalloprotease enzymes, such as MMP2 or MMP9. SCUBE3-C consists of 3 cysteine-rich domains and one CUB domain (Complement proteins C1r/C1s, Uegf and Bmp1). In another aspect, the activator of hair growth is a combination of pure recombinant SCUBE3 with one recombinant MMP enzyme, such as MMP2 or MMP9. In another aspect, the activator of hair growth is one of several synthetic peptides (SCUBE3-PP). One such SCUBE3-PP contains amino acid sequence of SCUBE3′s 3 cysteine-rich domains and one CUB domain. Other SCUBE3-PP can be: (a) SCUBE3 CUB+one cysteine-rich domain; (b) SCUBE3 CUB+two cysteine-rich domains. Also described herein is a method of intradermal microinjection of recombinant SCUBE3, SCUBE-C, SCUBE3+MMP or one of SCUBE3-PP to promote hair growth. In another aspect, described is a method of topical skin delivery of recombinant SCUBE3, SCUBE-C, SCUBE3+MMP or one of SCUBE3-PP to promote hair growth following increase in skin permeability, such as with micro-needling.

### Definitions

All scientific and technical terms used in this application have meanings commonly used in the art unless otherwise specified. As used in this application, the following words or phrases have the meanings specified.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of expressing the encoded protein.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “complement” and “complementary” as used herein, refers to the ability of two DNA molecules to base pair with each other, where an adenine on one DNA molecule will base pair to a thymine on a second DNA molecule and a guanine on one DNA molecule will base pair to a cytosine on a second DNA molecule. Two DNA molecules are complementary to each other when a nucleotide sequence in one DNA molecule can base pair with a nucleotide sequence in a second DNA molecule. For instance, the two DNA molecules 5′-ATGC and 5′-GCAT are complementary, and the complement of the DNA molecule 5′-ATGC is 5′-GCAT. The term complement and complementary also encompasses two DNA molecules where one DNA molecule contains at least one nucleotide that will not base pair to at least one nucleotide present on a second DNA molecule. For instance, the third nucleotide of each of the two DNA molecules 5′-ATTGC and 5′-GCTAT will not base pair, but these two DNA molecules are complementary as defined herein. Typically, two DNA molecules are complementary if they hybridize under the standard conditions referred to above. Typically, two DNA molecules are complementary if they have at least about 80% sequence identity, preferably at least about 90% sequence identity.

The term “modified” (or “chemically modified”) refers to the derivatization of a polypeptide with one or more moieties by appending (e.g., attaching via covalent or non-covalent interactions) one or more moieties to one or more amino acid residues of that polypeptide. Exemplary modifications include hydrophobic moieties such as lipophilic moieties and fatty acid moieties, glycosylation, phosphorylation. Further exemplary modifications include hydrophilic modifications. Polypeptides for use in the methods described herein, including SCUBE polypeptides, can be modified. Modified polypeptides for use in the methods retain one or more of the biological activities of the native polypeptide, and preferably additional possess one or more advantageous physiochemical properties in comparison to the corresponding native and/or un-modified polypeptide. For modified or unmodified SCUBE polypeptides, bioactive fragments thereof, or other polypeptides for use in the methods described herein, preferred modified polypeptides or fragments retain the biological activity of the native polypeptide and preferably possess one or more advantageous physiochemical activity.

The term “appended” refers to the addition of one or more moieties to an amino acid residue. The term refers, without limitation, to the addition of any moiety to any amino acid residue. The term includes attachment of a moiety via covalent or non-covalent interactions.

The term “N-terminal amino acid residue” refers to the first amino acid residue (amino acid number 1) of a polypeptide or peptide.

The term “C-terminal amino acid residue” refers to the last amino acid residue (amino acid number n, wherein n=the total number of residues in the peptide or polypeptide) of a polypeptide or peptide.

In certain embodiments, the amino acids used in the application of this disclosure are those naturally occurring amino acids found in proteins, or the naturally occurring anabolic or catabolic products of such amino acids which contain amino and carboxyl groups, and their corresponding R groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group).

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds includes asymmetric carbon atoms. It is to be understood accordingly that the isomers arising from such asymmetry are included within the scope of this disclosure. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

Certain compounds of the present disclosure may exist in particular geometric or stereoisomeric forms. The present disclosure contemplates all such compounds, including cis-and trans-isomers, R- and S-enantiomers, diastereomers, (D)-isomers, (L)-isomers, the racemic mixtures thereof, and other mixtures thereof, as falling within the scope of this disclosure. Additional asymmetric carbon atoms may be present in a substituent such as an alkyl group. All such isomers, as well as mixtures thereof, are intended to be included in this disclosure.

If, for instance, a particular enantiomer of a compound of the present disclosure is desired, it may be prepared by asymmetric synthesis, or by derivation with a chiral auxiliary. where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

For purposes of this disclosure, the chemical elements are identified in accordance with the Periodic Table of the Elements, CAS version, Handbook of Chemistry and Physics, 67th Ed., 1986-87, inside cover. Also for purposes of this disclosure, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Proteins, Peptides, and Compositions

Described herein are compositions comprising one or more proteins or peptides for stimulating hair growth; and, optionally, a pharmaceutically acceptable excipient. In some embodiments, the composition comprises a Signal Peptide, CUB Domain and EGF Like Domain Containing (SCUBE) protein. In some embodiments, the composition comprises a C-terminus peptide thereof (SCUBE-C). In some embodiments, the composition comprises a synthetic SCUBE peptide (SCUBE-PP). In some embodiments, the composition comprises a nucleic acid sequence encoding SCUBE, SCUBE-C, or SCUBE-PP. In some embodiments, the SCUBE protein is a SCUBE3 protein. In some embodiments, the SCUBE3 protein has the amino acid sequence shown in SEQ ID NO: 1. In some embodiments, the C-terminus peptide of SCUBE is a SCUBE3-C having the amino acid sequence shown in SEQ ID NO: 2. In some embodiments, the synthetic SCUBE peptide comprises a synthetic SCUBE3 peptide (SCUBE3-PP).

In some embodiments, the composition comprises a nucleic acid sequence encoding SCUBE3, SCUBE3-C, or SCUBE3-PP. In some embodiments, the nucleic acid sequence further comprises a promoter and/or other regulatory elements in operable linkage with the SCUBE protein or peptide encoding sequence.

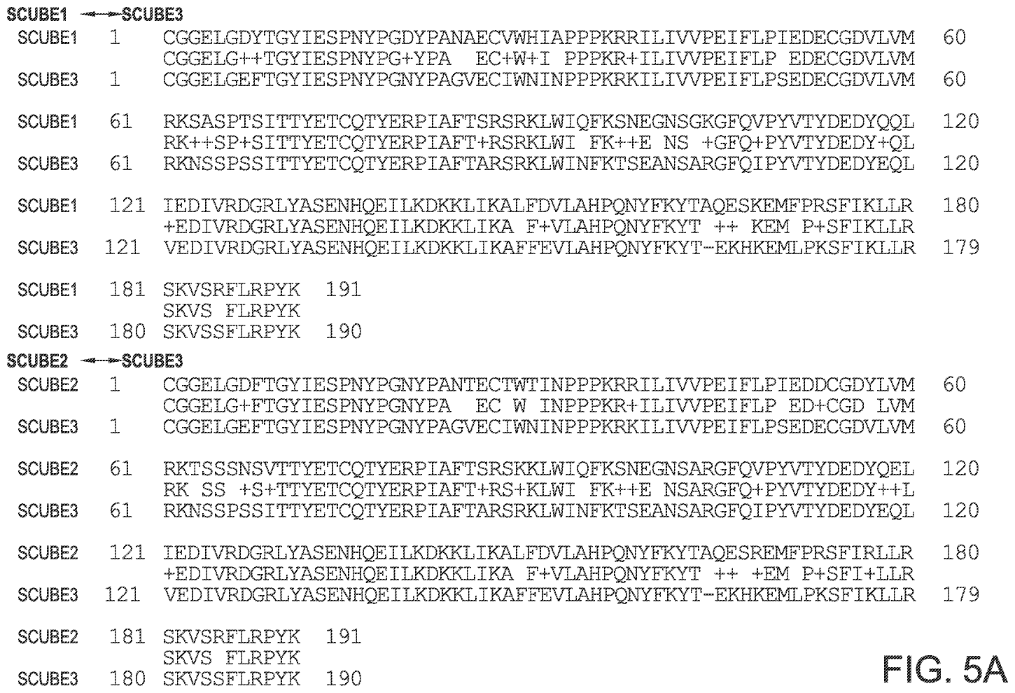

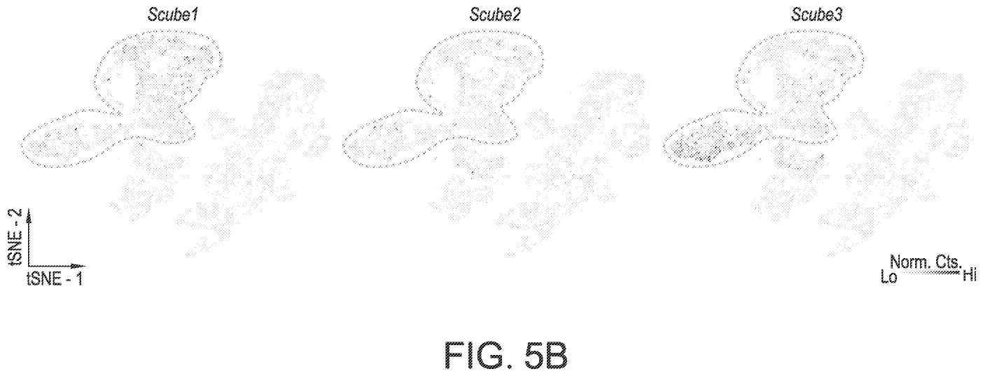

Other examples of a SCUBE protein include SCUBE1 (Protein Accession: Q8IWY4; SEQ ID NO: 3) and SCUBE2 (Protein Accession: Q9NQ36; SEQ ID NO: 5). SCUBE1 and SCUBE2 exhibit over 60% homology with SCUBE3 over its full length, and 83.25% and 82.20% amino acid homology, respectively, in the CUB domain (Wu et al., 2004, J Biol Chem 279, 37485-37490; FIG. 5A). SCUBE1 in particular, and SCUBE2 to a lesser degree, are expressed in the same hair follicle components as SCUBE3. Use of SCUBE1, SCUBE2, and/or SCUBE3 proteins or peptides (e.g., SCUBE1-C (SEQ ID NO: 4), SCUBE1-PP. SCUBE2-C (SEQ ID NO: 6), SCUBE2-PP) is contemplated, optionally in combination.

In some embodiments, the SCUBE3-C comprises 3 cysteine-rich domains and one CUB domain (Complement proteins C1r/C1s, Uegf and Bmp 1). In some embodiments, the SCUBE3-C peptide is produced by proteolytic cleavage of SCUBE3 with a protease. In some embodiments, the SCUBE3-C peptide is produced synthetically. In some embodiments, the protease is one or more matrix metalloprotease (MMP) enzymes. In some embodiments, the MMP enzyme is synthetic. In some embodiments, the composition further comprises an MMP enzyme. In some embodiments, the MMP enzyme is selected from MMP2 and MMP9. In some embodiments, the SCUBE3-PP comprises 3 cysteine-rich domains and a CUB domain, a CUB domain and one cysteine-rich domain; or a CUB domain and two cysteine-rich domains.

In some embodiments, the 3 cysteine-rich domains have the amino acid sequence: GTKCVSCP QGTYYHGQTE QCVPCPAGTF QEREGQLSCD LCPGSDAHGP LGATNVTTCA GQCPPGQHSV DGFKPCQPCP RGTYQPEAGR TLCFPCGGGL TTKHEGAISF QDCDTKVQCS PGHYYNTSIH RCIRCAMGSY QPDFRQNFCS RCPGNTSTDF DGSTSVAQCK NRQ (SEQ ID NO: 7). In some embodiments, the CUB domain has the amino acid sequence: CGGELGE FTGYIESPNY PGNYPAGVEC IWNINPPPKR KILIVVPEIF LPSEDECGDV LVMRKNSSPS SITTYETCQT YERPIAFTAR SRKLWINFKT SEANSARGFQ IPYVTYDEDY EQLVEDIVRD GRLYASENHQ EILKDKKLIK AFFEVLAHPQ NYFKYTEKHK EMLPKSFIKL LRSKVSSFLR PYK (SEQ ID NO: 2).

### Methods

Described herein are methods for promoting hair growth in a subject. Additionally provided is a method of treating alopecia in a subject. In some embodiments, these methods comprise intradermal microinjection of a SCUBE composition as described herein. In some embodiments, these methods comprise topical skin administration of a SCUBE composition as described herein. In some embodiments, the topical skin administration is provided following an increase in skin permeability. In some embodiments, the increase in skin permeability occurs by micro-needling.

The SCUBE protein or peptide to be administered in the methods is typically provided in pure form, for example, as a composition that is substantially free of other proteins or peptides. In some embodiments, the composition is at least 90% SCUBE protein or peptide, or at least “90% pure”. In some embodiments, the composition is at least 95%, 96%, 97%, 98%, or 99% pure SCUBE protein or peptide. Likewise, when administered in nucleic acid form, for example, as DNA or RNA, the composition is similarly free of non-SCUBE nucleotides. Such nucleotide-based compositions are optionally delivered in a lipid preparation. In some embodiments, the SCUBE protein or peptide is provided in recombinant or synthetic form, optionally with substitutions or modifications to enhance the stability or efficacy of the peptide. The pure form of SCUBE protein or peptide can comprise SCUBE3, SCUBE3-C, or SCUBE-3PP to the substantial exclusion of other SCUBE proteins or peptides. In some embodiments, the pure form of SCUBE protein or peptide comprises a mixture of SCUBE1, SCUBE2, and SCUBE3 proteins and/or peptides.

In some embodiments, the SCUBE protein or peptide is delivered in the form of beads or other known delivery vehicle. In some embodiments, the SCUBE protein or peptide is administered via wearable patches that provide extended release.

In some embodiments, the alopecia is androgenic alopecia, female pattern hair loss, alopecia areata, traction alopecia, alopecia totalis, cicatricial alopecia, and/or alopecia universalis. The alopecia can be what is known as male pattern baldness, or the more diffuse type of hair loss often observed in postmenopausal women. In some embodiments, the method generates new hair follicles, accelerates hair regrowth after a hair graft, chemotherapy, and/or increases the size of existing hair follicles. For example, the SCUBE composition can be administered about one month after a transplantation to accelerate the regrowth of hair while the transplanted follicles are re-established. Likewise, the SCUBE composition can be administered after chemotherapy to facilitate regrowth of hair.

### Kits

Described herein are kits comprising a set of agents as described herein, such as proteins, peptides, nucleic acid molecules, and vectors, and optionally, one or more suitable containers containing such agents. Agents can optionally include a detectable label or means for monitoring or regulating the agent. Labels can be fluorescent, luminescent, enzymatic, chromogenic, or radioactive.

The kit will typically comprise the container(s) described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use, such as those described herein. Directions and or other information can also be included on an insert, which is included with the kit.

### EXAMPLES

The following examples are presented to illustrate the present disclosure and to assist one of ordinary skill in making and using the same. The examples are not intended in any way to otherwise limit the scope of the disclosure.

### Example 1: Hedgehog Signaling Reprograms Hair Follicle Niche Fibroblasts to a Hyper-Activated State

This Example demonstrates that SCUBE3 is a new mesenchymal niche factor that activates hair growth. Dermal papilla (DP) is the key mesenchymal niche cell type, that regulates cyclic regeneration of hair follicles by orchestrating paracrine signaling crosstalk with epithelial progenitors. We discovered that leptin receptor (Lepr) is a robust marker gene of adult DP fibroblasts in mice and that constitutive Cre and inducible CreER genetic tools, whose activity follows the expression of the Lepr isoform B, can efficiently target DPs for labeling and gene modulation during both growth (anagen) and rest (telogen) phases of the hair growth cycle. Using Lepr-based DP targeting, we show that Hedgehog signaling activation in adult DPs prominently accelerates cyclic growth of hair follicles and induces formation of new hair follicles from the sides of pre-existing follicles in unwounded skin. By single-cell RNA-sequencing we fully define cellular heterogeneity of DP fibroblasts, that increases upon Hedgehog activation, and establish new gene regulatory network of hyper-activated DP state. We also identify new Hedgehog-regulated signaling factors, Scube3 and reveal its role in physiological growth of hair follicles. In normal skin, Scube3 is expressed only in dermal papilla fibroblasts of growing, but not resting follicles. Scube3 null mice showed anagen initiation delay. SCUBE3 protein microinjection is sufficient to induce hair growth. Moreover, dermal papilla-enriched expression of SCUBE3 and its growth activating effect are partially conserved in human scalp hair follicles.

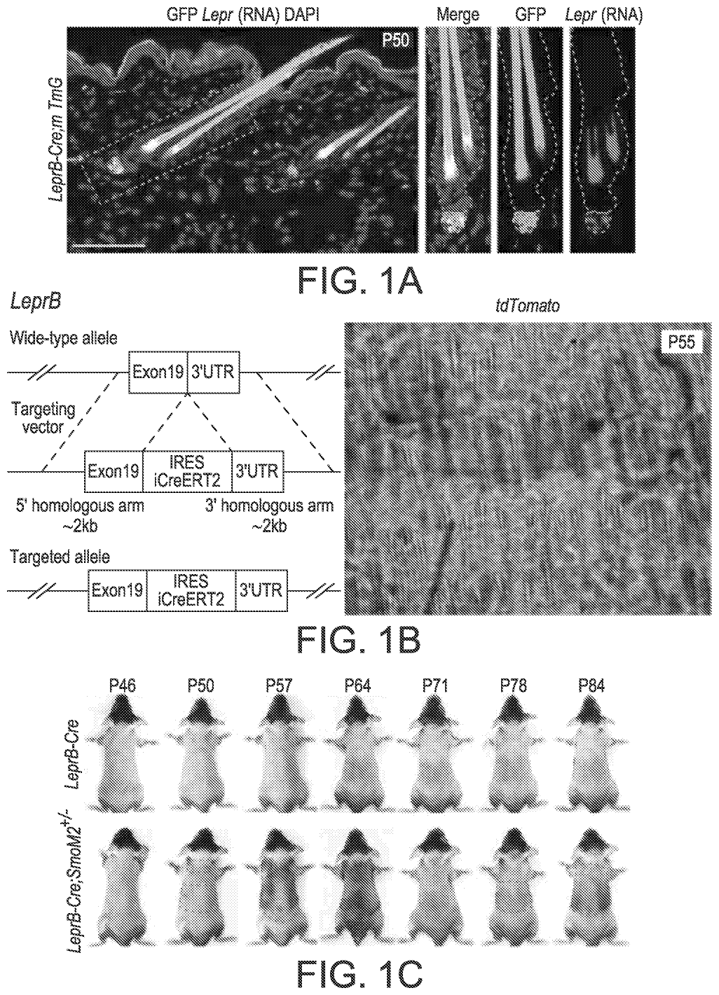

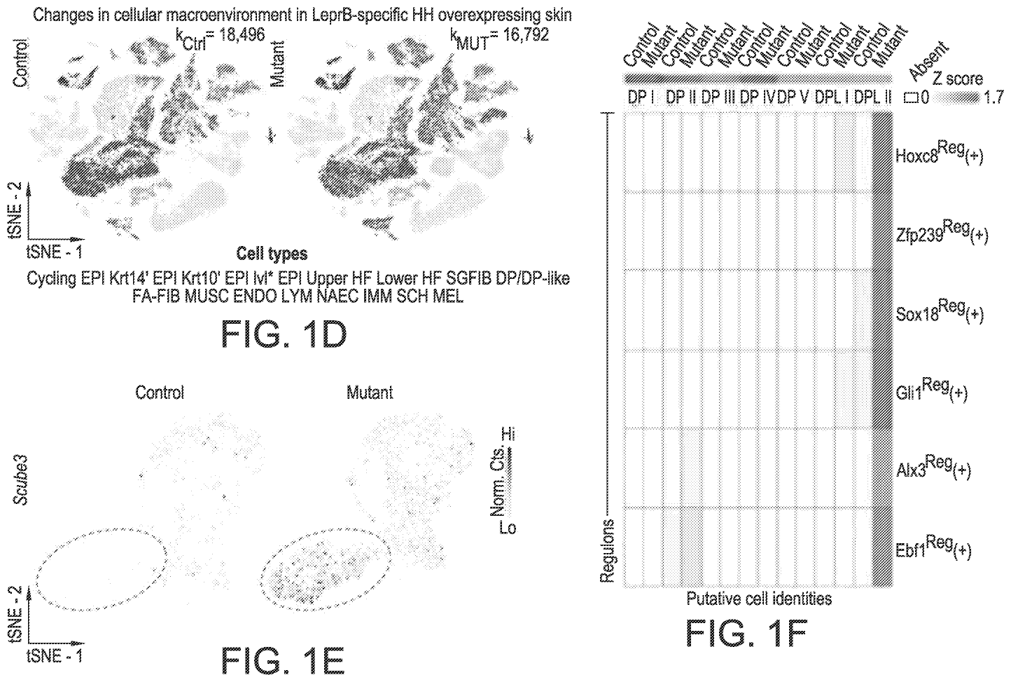

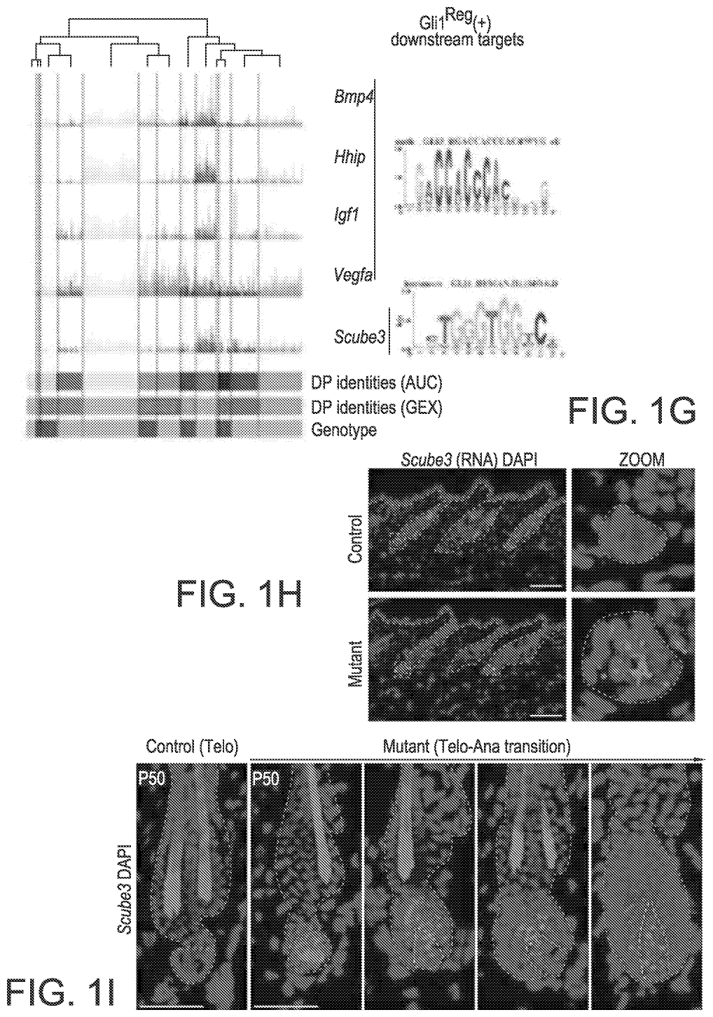

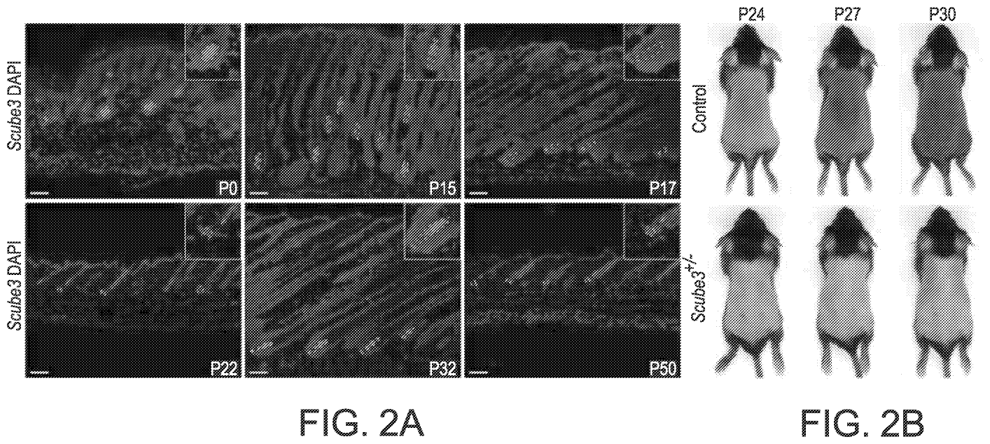

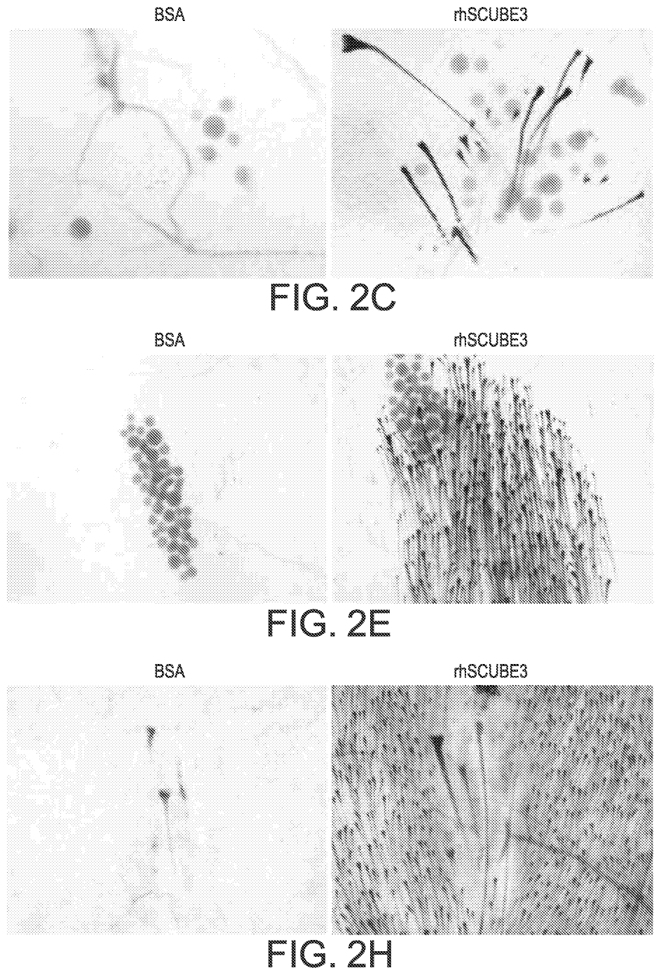

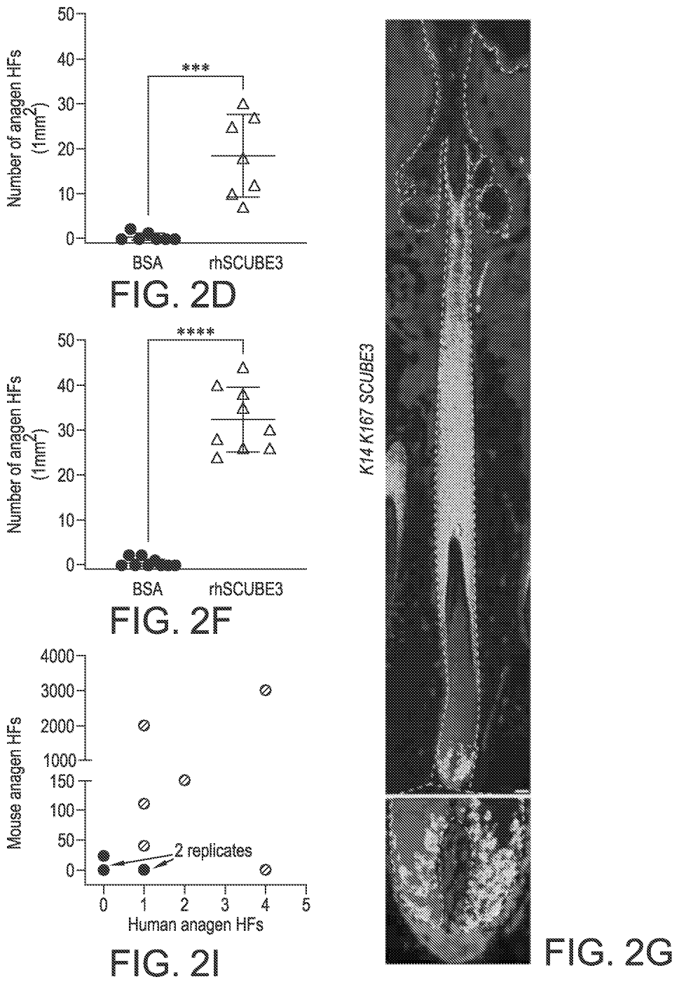

To identify new Cre recombinase driver for DP cells, and to be able to genetically manipulate these cells in the context of hair growth, we examined the previously reported LeprB-Cre mouse line, where an IRES-Cre recombinase expression cassette is placed into the 3′ untranslated region of Lepr, allowing for Cre activity to be restricted to cells transcribing LeprB isoform (DeFalco et al., 2001). We characterized labeling pattern in dorsal skin of LeprB-Cre;mTmG reporter mice. DP labeling progressively increased by late first anagen, became high yet incomplete during first telogen and second anagen at P22 and P32, respectively, and eventually became high and nearly complete by second telogen at P50 (FIG. 1A). RNAscope further confirmed expression of pan-isoform Lepr in GFP+DP fibroblasts in P50 LeprB-Cre;mTmG skin (FIG. 2A). To enable inducible labeling and targeting of DPs based on their expressing LeprB isoform, we generated new LeprB-CreER transgenic mice in which IRES-iCreERT2 construct was inserted after exon 19 stop codon of Lepr gene (FIG. 1B, left). To test its specificity and efficacy, we treated LeprB-CreER;tdTomato mice with five daily doses of tamoxifen starting at P50. When skin was examined after one additional day, LeprB-CreER labeled DPs prominently and specifically, with virtually no extra-follicular cell targeting (FIG. 1B, right). Thus, we conclude that in dorsal mouse skin, constitutive LeprB-Cre most efficiently targets DPs, with labeling prominently increasing during the second hair growth cycle, and that inducible LeprB-CreER can permit specific and efficient DP targeting in adult mouse skin.

We asked if inducing constitutive Hedgehog signaling in dermal cells using LeprB-Cre can affect HF growth. Normally, Hedgehog is required for embryonic hair development (Chiang et al., 1999; Gritli-Linde et al., 2007; Mill et al., 2003; St-Jacques et al., 1998; Woo et al., 2012) and for proper hair growth activation (Hsu et al., 2014; Oro and Higgins, 2003; Paladini et al., 2005; Sato et al., 1999). A recent study documented that inducing Hedgehog signaling in myofibroblasts by means of constitutively active Smoothened (SmoM2) overexpression results in regeneration of many neogenic HFs in small wounds of adult mice that would otherwise heal with a hairless scar (Lim et al., 2018). Concomitant activation of Hedgehog signaling in wound epidermis is not required for this regenerative event. In contrast, in adult unwounded skin, concomitant epidermal and dermal Hedgehog activation by means of Ptch1 deletion was reported to be necessary to induce HF neogenesis. Neogenic HFs formed from pre-existing HFs in hairless paw skin and from touch domes—specialized sensory structures in the interfollicular epidermis (Sun et al., 2020). Hedgehog activation by means of Ptch1 deletion in Col1a2-CreER-expressing dermal cells alone was not sufficient to induced HF neogenesis in unwounded skin and, instead, led to an increase in fibroblast density. We generated LeprB-Cre; SmoM2+/−mutant mice and compared their hair phenotypes to those of LeprB-Cre control mice. Hair growth was traced non-invasively via periodic hair clipping and photography as previously described (Plikus et al., 2008; Wang et al., 2017). Consistent with a relatively late onset of LeprB-Cre activation in DP fibroblasts (FIG. 1C), mutant mice did not show obvious hair growth cycle difference until second telogen. However, while no new hair growth was observed in control mice (n=8) during the entire observation period spanning second telogen days P46 through P86, mutant mice (n=8) showed prominently precocious hair growth (FIG. 1C).

To identify candidate cell types and molecular events that drive enhanced hair growth downstream of Hedgehog activation, we profiled dorsal skin of LeprB-Cre;SmoM2+/−; mTmG mutant and LeprB-Cre;mTmG control mice by scRNA-seq at P46, a time point that precedes the appearance of mutant hair phenotype by a few days. A total of 18,496 control (n=2) and 16,792 mutant (n=2) sequenced cells met quality control-filtering metrics and were processed for downstream analysis. Upon unsupervised clustering, all cells grouped into seventeen main clusters and were assigned cell identities according to differential expression of signature genes. These cell types included interfollicular and hair follicle keratinocytes, sebocytes, dermal, fibro-adipogenic and DP fibroblasts, Schwann cells, melanocytes, immune cells, vascular, lymphatic and neuron-associated endothelial cells, and muscle cells (FIGS. 1D). Prominent enrichment for mutant cells were observed among fibroblasts, where some DP clusters were almost entirely composed of mutant cells (FIGS. 1D, 1E). This indicated that LeprB-Cre; SmoM2+/−hair phenotypes are likely driven by newly formed, Hedgehog-active DP fibroblasts (DPL fibroblasts). Next, we performed differential gene expression analysis comparing mutant DP clusters (including DPL fibroblasts) vs.

control DP clusters using DESeq2. Prominent change of Scube3 expression was identified, which was driven by DPL cells becoming new cellular source for a non-classical activating ligand Scube3 (FIGS. 1E, 1H). At P50, LeprB-Cre;SmoM2+/−HFs, whose morphology ranged from telogen-like to early anagen-like, showed prominent Scube3 expression in DP cells, HG cells and their derivatives (FIGS. 1I, right). In contrast, control HFs remained in telogen with absent Scube3 expression (FIGS. 1I, left). We then performed Gene Regulatory Network (GRN) analysis to explore regulon activity profile in the mutant DPL fibroblasts using pySCENIC (Van de Sande et al., 2020). Confirming transgenic stimulation of Hedgehog signaling, mutant DPL II cells strongly activated Gli1Reg(+) regulon (FIGS. 1F). Other top activated regulons included Hoxc8Reg(+), Zfp239Reg(+), Sox18Reg(+), Alx3Reg(+), and Ebf1Reg(+) (FIGS. 1F). Further, several signaling genes upregulated in DPL fibroblasts, including Scube3, Bmp4, Hhip, Igf1, and Vegfa, were predicted to be downstream of Gli1Reg(+) regulon (FIGS. 1G). Thus, mutant DPL fibroblasts activate redundant GRNs downstream of transgenically stimulated Gli1Reg (+), including Scube3 gene, which potentially promotes hair growth in the mutant mice.

SCUBE3 is a multifunctional protein that can augment BMP (Lin et al., 2021) and FGF8 signaling (Tu et al., 2014) on source cells in its membrane-tethered form, or TGFβ signaling in its proteolytically cleaved secreted form (Wu et al., 2011; Yang et al., 2007;Yang et al., 2020). In embryonic mouse skin, Scube3 is preferentially expressed in DP and DS compartments of developing HFs (Haworth et al., 2007). Yet, the role of SCUBE3 in regulating hair growth remains an enigma. To elucidate the role of Scube3 in hair cyclic growth, we then profiled Scube3 expression pattern using RNAscope (FIG. 2A). Scube3 was highly expressed in DPs of P0 and P5 early anagen HFs, but then prominently decreased by P17, ahead of anagen termination. It was absent in first telogen HFs at P22, reappeared in second anagen HFs at P32 and then turned off again during second telogen at P50. Scattered expression was also seen in hair matrix but only during first anagen phase. Thus, Scube3 is anagen phase-associated DP marker gene. We also examined timing of second anagen initiation in dorsal skin of germline Scube3 null mice, that are viable and develop grossly normal fur, despite having distinct craniofacial defects (Lin et al., 2021). Compared to littermate controls, Scube3 null mice show distinct, albeit mild anagen initiation delay (FIG. 2B), suggesting that Scube3 is indispensable for anagen hair growth.

Considering the above pattern and its overexpression in mutant DPL fibroblasts, we asked if SCUBE3 might exert anagen-promoting effect. When intradermally microinjected for four days starting at P48, recombinant human SCUBE3 (rhSCUBE3) induced significant new hair growth when compared on day 14 to microinjected bovine serum albumin (BSA) (mean difference=18+/−3.44; n=7) (FIG. 2C-D). Aging in mice is associated with HF miniaturization (Matsumura et al., 2016) and increase in extra-follicular hair growth inhibitors (Chen et al., 2014). To test if rhSCUBE3 can induce hair growth in aged skin, we repeated protein microinjection experiments in mice over 20 months of age. Unlike BSA, rhSCUBE3 potently stimulated new hair growth at the injection sites (mean difference=31.78+/−2.40; n=9) (FIG. 2E-F).

To start evaluating the translational potential of SCUBE3, first we examined its expression pattern in human anagen HFs from occipital scalp of volunteer donors, undergoing autologous hair transplantation procedures. On RNAscope, SCUBE3 is expressed in DP cells and strongly in the dermal sheath cup, a lowermost portion of DS that physically connects to DP and surrounds hair matrix (FIG. 2G). We also xenografted human occipital scalp HFs to pigmented SCID mice and treated them with three daily doses of rhSCUBE3 or BSA on post-grafting day 30, when grafted human HFs enter telogen (Oh et al., 2016). Anagen phase human and adjacent mouse host HFs were then counted at each injection site on post-grafting day 50. Compared to BSA (n=5), rhSCUBE3 (n=6) accelerated anagen entry of grafted human HFs and stimulated new anagen of mouse host HFs (FIG. 2H-I). We conclude that SCUBE3 is a novel mesenchymal niche-derived hair growth activator in mice, whose expression pattern and function are partially conserved in human scalp.

### REFERENCES

Chen, C.C., et al. (2014). J Invest Dermatol 134, 2086-2096.
Chiang, C., et al. (1999). Dev Biol 205, 1-9.
DeFalco, J., et al. (2001). Science 291, 2608-2613.
Gritli-Linde, A., et al. (2007). Dev Cell 12, 99-112.
Haworth, K., et al. (2007). Gene Expr Patterns 7, 630-634.
Hsu, Y.C., Li, L., and Fuchs, E. (2014). Cell 157, 935-949.
Lim, C.H., et al. (2018). Nat Commun 9, 4903.
Lin, Y.C., et al. (2021). Am J Hum Genet 108, 115-133.
Matsumura, H., et al. (2016). Science 351, aad4395.
Mill, P., et al. (2003). Genes Dev 17, 282-294.
Oh, J.W., et al. (2016). J Invest Dermatol 136, 34-44.
Oro, A.E., and Higgins, K. (2003). Dev Biol 255, 238-248.
Paladini, R.D., et al. (2005). J Invest Dermatol 125, 638-646.
Plikus, M.V., et al. (2008). Nature 451, 340-344.
Sato, N., Leopold, P.L., and Crystal, R.G. (1999). J Clin Invest 104, 855-864.
St-Jacques, B., et al. (1998). Curr Biol 8, 1058-1068.
Sun, X., et al. (2020). Elife 9.
Tu, C.F., et al. (2014). J Biol Chem 289, 18928-18942.
Van de Sande, B., et al. (2020). Nat Protoc 15, 2247-2276.
Wang, Q., et al. (2017). Elife 6.
Woo, W.M., Zhen, H.H., and Oro, A.E. (2012). Genes Dev 26, 1235-1246.
Wu, Y.Y., et al. (2011). Oncogene 30, 3682-3693.
Yang, H.Y., et al. (2007). Cardiovasc Res 75, 139-147.
Yang, X., et al. (2020). Cancer Biother Radiopharm 35, 120-128.

### Example 2: Validation of SCUBE3 Effect on in Vivo Hair Growth

The Scube3 flox/flox and the TetO-Scube3 mouse lines were generated for use in validating the effect of tissue specific Scube3 deletion/overexpression on hair growth in vivo.

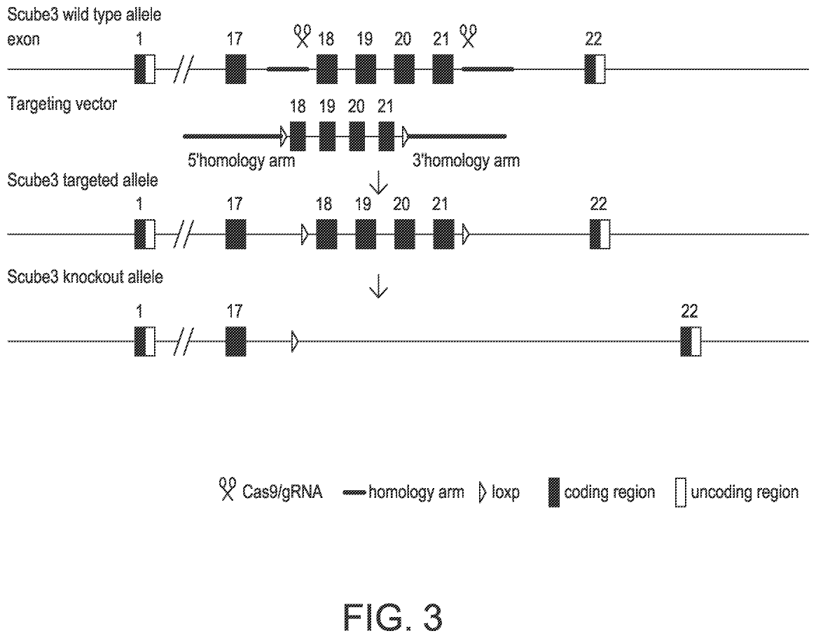

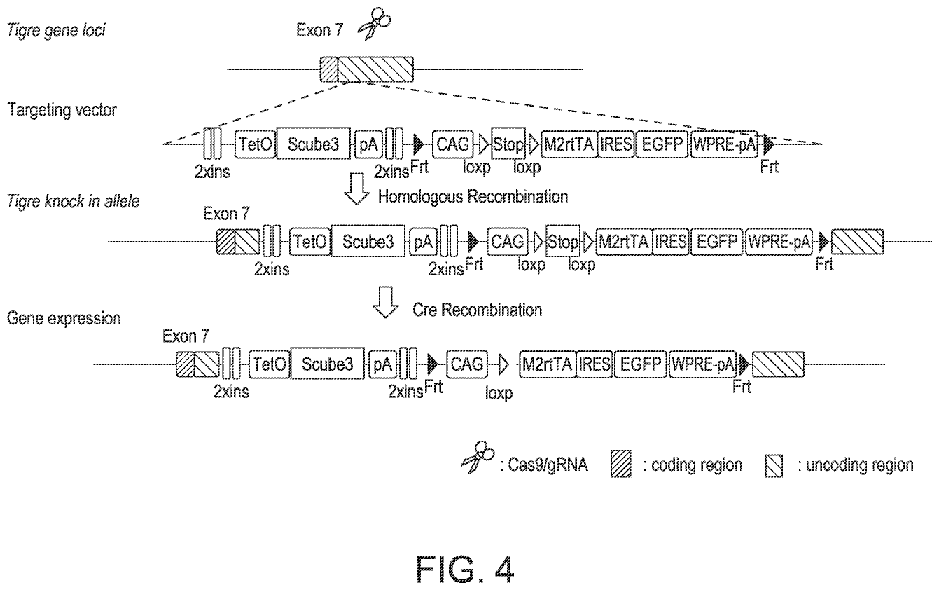

Mouse design drawings are shown in FIGS. 3 (Scube3 flox/flox mouse) and 4 (TetO-Scube3 mouse). We will cross the new flox and Tet-On transgenic mice with LeprB-CreER mice (generated in our recent study Liu Y, et al. Dev Cell. 2022 Jul. 25;57 (14): 1758-1775; PMID: 35777353). DP-specific Scube3 deletion in LeprB-CreER; Scube3foxfox (flox mutant) mice and Scube3 overexpression in LeprB-Cre; TetOn-Scube3 (TetOn mutant) mice will be achieved by tamoxifen induction in those mice. We will evaluate and compare hair growth patterns in flox and TetOn mutant mice and their control littermates, using well-established non-invasive hair cycle tracing assay, plucking-induced hair growth model, and immunostaining of skin for hair-growth markers, including proliferation markers. This will further validate our finding that SCUBE3 can induce hair growth in vivo. An example that supports a predictive relationship between overexpression/deletion in vivo and local injection/topical application/delivery can be found in Oshimori N, et al. Cell Stem Cell. 2012 Jan. 6;10 (1): 63-75.

Throughout this application various publications are referenced. The disclosures of these publications in their entireties are hereby incorporated by reference into this application in order to describe more fully the state of the art to which this disclosure pertains.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the invention as set forth in the appended claims.

1. A composition comprising a Signal Peptide, CUB Domain and EGF Like Domain Containing 3 (SCUBE3) protein; a C-terminus peptide thereof (SCUBE3-C); a synthetic SCUBE3 peptide (SCUBE3-PP); or a nucleic acid sequence encoding SCUBE3, SCUBE3-C, or SCUBE3-PP; and, optionally, a pharmaceutically acceptable excipient.

2. The composition of claim 1, wherein the SCUBE3-C comprises 3 cysteine-rich domains and one CUB domain (Complement proteins C1r/C1s, Uegf and Bmp1).

3. The composition of claim 2, wherein the SCUBE3-C peptide is produced by proteolytic cleavage of SCUBE3 with a protease.

4. The composition of claim 3, wherein the protease is one or more matrix metalloprotease (MMP) enzymes.

5. The composition of claim 1, further comprising an MMP enzyme.

6. The composition of claim 4, wherein the MMP enzyme is selected from MMP2 and MMP9.

7. The composition of claim 1, wherein the SCUBE3-PP comprises:
(a) 3 cysteine-rich domains and a CUB domain,
(b) a CUB domain and one cysteine-rich domain; or
(c) a CUB domain and two cysteine-rich domains.

8. The composition of claim 7, wherein the 3 cysteine-rich domains of (a) have the amino acid sequence: GTKCVSCP QGTYYHGQTE QCVPCPAGTF QEREGQLSCD LCPGSDAHGP LGATNVTTCA GQCPPGQHSV DGFKPCQPCP RGTYQPEAGR TLCFPCGGGL TTKHEGAISF QDCDTKVQCS PGHYYNTSIH RCIRCAMGSY QPDFRQNFCS RCPGNTSTDF DGSTSVAQCK NRQ (SEQ ID NO: 7).

9. The composition of claim 7, wherein the CUB domain has the amino acid sequence: CGGELGE FTGYIESPNY PGNYPAGVEC IWNINPPPKR KILIVVPEIF LPSEDECGDV LVMRKNSSPS SITTYETCQT YERPIAFTAR SRKLWINFKT SEANSARGFQ IPYVTYDEDY EQLVEDIVRD GRLYASENHQ EILKDKKLIK AFFEVLAHPQ NYFKYTEKHK EMLPKSFIKL LRSKVSSFLR PYK (SEQ ID NO: 2).

10. A method of promoting hair growth in a subject, the method comprising intradermal microinjection of a composition of claim 1.

11. A method of promoting hair growth in a subject, the method comprising topical skin administration of a composition of claim 1.

12. The method of claim 11, wherein the topical skin administration is provided following an increase in skin permeability.

13. The method of claim 12, wherein the increase in skin permeability occurs by micro-needling.

14. The method of claim 10, wherein the subject is a mammalian subject.

15. The method of claim 14, wherein the subject is human.

16. The method of claim 15, wherein the subject suffers from alopecia.

17. The method of claim 16, wherein the alopecia is androgenic alopecia, female pattern hair loss, alopecia areata, traction alopecia, alopecia totalis, cicatricial alopecia, and/or alopecia universalis.

18. The method of claim 15, wherein the method generates new hair follicles, accelerates hair regrowth after a hair transplantation, chemotherapy, and/or increases the size of existing hair follicles.

19. The composition of claim 1, wherein the SCUBE3-PP is chemically modified.

20. The method of claim 10, wherein the SCUBE3-PP is chemically modified.


SEQUENCE LISTING (7 sequences)

SEQ ID NO: 1 - protein, 993 aa, Homo sapiens
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPESENGFTVSCGTPSPRAAPARAGHNGNSTNSNHCHEAAVLSIKQRASFKIKDAKCRLHLRNKGKTEEAGRITGPGGAPCSECQVTFIHLKCDSSRKGKGRRARTPPGKEVTRLTLELEAEVRAEETTASCGLPCLRQRMERRLKGSLKMLRKSINQDRFLLRLAGLDYELAHKPGLVAGERAEPMESCRPGQHRAGTKCVSCPQGTYYHGQTEQCVPCPAGTFQEREGQLSCDLCPGSDAHGPLGATNVTTCAGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQCGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 2 - protein, 190 aa, Homo sapiens
CGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 3 - protein, 988 aa, Homo sapiens
MGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPTCGCHQKYALHSDGRTCIETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNGKDCVETGKCLSRAKTSPRAQLSCSKAGGVESCFLSCPAHTLFVPDSENSYVLSCGVPGPQGKALQKRNGTSSGLGPSCSDAPTTPIKQKARFKIRDAKCHLRPHSQARAKETARQPLLDHCHVTFVTLKCDSSKKRRRGRKSPSKEVSHITAEFEIETKMEEASDTCEADCLRKRAEQSLQAAIKTLRKSIGRQQFYVQVSGTEYEVAQRPAKALEGQGACGAGQVLQDSKCVACGPGTHFGGELGQCVSCMPGTYQDMEGQLSCTPCPSSDGLGLPGARNVSECGGQCSPGFFSADGFKPCQACPVGTYQPEPGRTGCFPCGGGLLTKHEGTTSFQDCEAKVHCSPGHHYNTTTHRCIRCPVGTYQPEFGQNHCITCPGNTSTDFDGSTNVTHCKNQHCGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDEDYQQLIEDIVRDGRLYASENHQEILKDKKLIKALFDVLAHPQNYFKYTAQESKEMFPRSFIKLLRSKVSRFLRPYK

SEQ ID NO: 4 - protein, 191 aa, Homo sapiens
CGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDEDYQQLIEDIVRDGRLYASENHQEILKDKKLIKALFDVLAHPQNYFKYTAQESKEMFPRSFIKLLRSKVSRFLRPYK

SEQ ID NO: 5 - protein, 999 aa, Homo sapiens
MGVAGRNRPGAAWAVLLLLLLLPPLLLLAGAVPPGRGRAAGPQEDVDECAQGLDDCHADALCQNTPTSYKCSCKPGYQGEGRQCEDIDECGNELNGGCVHDCLNIPGNYRCTCFDGFMLAHDGHNCLDVDECLENNGGCQHTCVNVMGSYECCCKEGFFLSDNQHTCIHRSEEGLSCMNKDHGCSHICKEAPRGSVACECRPGFELAKNQRDCILTCNHGNGGCQHSCDDTADGPECSCHPQYKMHTDGRSCLEREDTVLEVTESNTTSVVDGDKRVKRRLLMETCAVNNGGCDRTCKDTSTGVHCSCPVGFTLQLDGKTCKDIDECQTRNGGCDHFCKNIVGSFDCGCKKGFKLLTDEKSCQDVDECSLDRTCDHSCINHPGTFACACNRGYTLYGFTHCGDTNECSINNGGCQQVCVNTVGSYECQCHPGYKLHWNKKDCVEVKGLLPTSVSPRVSLHCGKSGGGDGCFLRCHSGIHLSSDVTTIRTSVTFKLNEGKCSLKNAELFPEGLRPALPEKHSSVKESFRYVNLTCSSGKQVPGAPGRPSTPKEMFITVEFELETNQKEVTASCDLSCIVKRTEKRLRKAIRTLRKAVHREQFHLQLSGMNLDVAKKPPRTSERQAESCGVGQGHAENQCVSCRAGTYYDGARERCILCPNGTFQNEEGQMTCEPCPRPGNSGALKTPEAWNMSECGGLCQPGEYSADGFAPCQLCALGTFQPEAGRTSCFPCGGGLATKHQGATSFQDCETRVQCSPGHFYNTTTHRCIRCPVGTYQPEFGKNNCVSCPGNTTTDFDGSTNITQCKNRRCGGELGDFTGYIESPNYPGNYPANTECTWTINPPPKRRILIVVPEIFLPIEDDCGDYLVMRKTSSSNSVTTYETCQTYERPIAFTSRSKKLWIQFKSNEGNSARGFQVPYVTYDEDYQELIEDIVRDGRLYASENHQEILKDKKLIKALFDVLAHPQNYFKYTAQESREMFPRSFIRLLRSKVSRFLRPYK

SEQ ID NO: 6 - protein, 191 aa, Homo sapiens
CGGELGDFTGYIESPNYPGNYPANTECTWTINPPPKRRILIVVPEIFLPIEDDCGDYLVMRKTSSSNSVTTYETCQTYERPIAFTSRSKKLWIQFKSNEGNSARGFQVPYVTYDEDYQELIEDIVRDGRLYASENHQEILKDKKLIKALFDVLAHPQNYFKYTAQESREMFPRSFIRLLRSKVSRFLRPYK

SEQ ID NO: 7 - protein, 171 aa, Homo sapiens
GTKCVSCPQGTYYHGQTEQCVPCPAGTFQEREGQLSCDLCPGSDAHGPLGATNVTTCAGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQ